(19) United States
(12) Patent Application Publication
(10) Pub. No.: US 2023/0211041 A1
(43) Pub. Date: Jul. 6, 2023

(54) Self-Sterilizing Wound Dressing

(71) Applicant: Kraton Polymers LLC, Houston, TX (US)

(72) Inventors: Vijay Mhetar, Houston, TX (US); Roger Tocchetto, Houston, TX (US)

(73) Assignee: Kraton Polymers LLC, Houston, TX (US)

(21) Appl. No.: 17/995,991

(22) PCT Filed: Apr. 19, 2021
(86) PCT No.: PCT/US2021/070414

§ 371 Date: Oct. 12, 2022

Related U.S. Application Data
(60) Provisional application No. 63/011,576, filed on Apr. 17, 2020.
(60) Provisional application No. 63/019,634, filed on May 4, 2020.
(60) Provisional application No. 63/200,302, filed on Feb. 28, 2021.

(51) Int. Cl. A61L 15/42; A61L 17/10; A61L 15/22; A61L 17/00

(52) U.S. Cl. CPC A61L 15/42; A61L 17/10; A61L 15/225; A61L 17/105; A61L 17/005; A61L 2300/404

(57) ABSTRACT

A self-sterilizing wound dressing is disclosed. The wound dressing comprises a substrate having a first surface facing at least a portion of a wound or a surgical site and a second surface facing opposite to the first surface. At least one surface of the substrate comprises a sulfonated polymer selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof. The sulfonated polymer is sufficiently or selectively sulfonated to contain from 10-100 mol % sulfonic acid or sulfonate salt functional groups based on the number of monomer units, for killing at least 90% of microbes in less than 120 minutes of coming into contact with the wound dressing.

### TECHNICAL FIELD

The disclosure relates to a wound dressing, having an antimicrobial layer disposed on at least one surface of the substrate, for killing microorganisms when applied on wound or surgical site.

### BACKGROUND

Conventional wound dressing to be used for treating a wound on the skin comes in different forms based on desired level of protection. In case of more serious wounds, the wound dressing involves multilayer assembly including layers of dressing materials, fluid absorption layers, and medicaments to treat the wound. Most of the commercially available dressings are based on hydrogels, hydrocolloids, semipermeable adhesive films, perforated films, alginates, polysaccharide beads, and polyurethane foams. Despite of different forms of wound dressings, healing efficiency of wound is hampered due to the infection caused by microorganisms resulting in a delayed treatment to cure the patient. To protect the skin having the wound from microbial infections, a simple antimicrobial solution is desired which can be easily accommodated in the wound dressing.

There is a need for a wound dressing assembly which can at least kill 95% microorganisms in a short period of time upon contact with part of the skin having the wound or the surgical site.

### SUMMARY

The disclosure is directed to a self-contained antimicrobial wound dressing. The wound dressing comprising: a substrate having a first surface facing at least a portion of a wound or a surgical site and a second surface facing opposite to the first surface. The first surface and/or the second surface of the substrate comprising a sulfonated polymeric layer having a thickness of at least >1μm, capable of killing at least 95% microorganisms and inhibiting growth of microorganisms. The sulfonated polymeric layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof. The sulfonated polymer has a degree of sulfonation of at least 10%.

In some aspects, the sulfonated polymeric layer comprises at least 50 wt. %, more preferably at least 70 wt. %, even more preferably at least 90 wt. %, yet more preferably at least 95 wt. %, still more preferably at least 98 wt. %, even more preferably at least 99 wt. % and most preferably 100 wt. % (i.e. consists) of one or more of the sulfonated polymers.

In one aspect, the sulfonated polymeric layer deposited on at least one surface of the substrate and capable of killing at least 95% microorganisms within 30 minutes of contact with the sulfonated polymeric layer.

In another aspect, the sulfonated polymeric layer is applied onto the first surface of the substrate by dip coating, spray coating, dispersion coating, solvent casting, or adhesively attached to the first surface of the substrate as a peel-and-stick film.

In still another aspect, the wound dressing comprising an adhesive layer coupled between the first surface of the substrate and the sulfonated polymeric layer. The adhesive layer helps in securing the wound dressing to the desired area surrounding the wound or surgical site.

### DETAILED DESCRIPTION

The following terms used in the specification have the following meanings:

“Wound” refers to a physical injury to living tissue caused by a cut, a blow, a lesion, a gash, a laceration, a tear, a puncture, a sore, a graze, a scratch, a scrape, an abrasion, a bruise, and a contusion, or any degradation of its normal structure and function resulting from an internal or external pathology that results in an opening or break of the skin. A healing wound has aspects relating to control of infection, resolution of inflammation, angiogenesis, regeneration of a functional connective tissue matrix, contraction, resurfacing, differentiation, and remodeling. The wound herein is taken to mean mammals such as sheep, horses, cattle, pigs, dogs, cats, rats, mice and primates, including humans.

“Wound dressing” refers to anything that is used in direct contact with a wound to help in healing and prevent further issues or complications. The wound dressing may include a dressing, a band-aid, an absorbent pad, a surgical article, a suture for stitching cuts or wounds, as they all aim to increase healing of the wound and decrease risks of infection. The wound dressing can be in the form of cloth dressings, foam dressings, gauze sponge, gauze bandage roll, non-adherent pads, non-adherent wet dressings, transparent dressings, hydrocolloid dressings, hydrogel dressings, calcium alginates, collagen dressings, and stitches.

“Substrate” refers to a layer under something else to provide support, having two surfaces available to deposit or coat a material of the choice.

“Effective amount” refers to an amount sufficient to alter, destroy, inactivate, neutralize and/or inhibit growth of microorganisms, e.g., an amount sufficient to sterilize and kill microorganisms in contact with the sulfonated polymeric layer applied onto at least one surface of the substrate of the wound dressing.

“Ion Exchange Capacity” or IEC refers to the total active sites or functional groups responsible for ion exchange in a polymer. Generally, a conventional acid-base titration method is used to determine the IEC, see for example International Journal of Hydrogen Energy, Volume 39, Issue 10, Mar. 26, 2014, Pages 5054-5062, “Determination of the ion exchange capacity of anion-selective membrane.” IEC is the inverse of “equivalent weight” or EW, which the weight of the polymer required to provide 1 mole of exchangeable protons.

“Microorganisms” refer to organisms with microscopic size including bacteria, archaea, fungi (yeasts and molds), algae, protozoa, and viruses including coronavirus.

“Peel-and-stick” or “peel-and-stick film” refers to a laminate having at least two layers, a release layer or liner which can also be a support layer, and another layer containing the sulfonated polymer. The peel-and-stick is self-adhesive, or releasable or peelable, or removable after being attached to a surface. The release layer is optionally coated with an adhesive which permits it to stick to a surface without glue, paste, or the like, allowing the peel-and-stick to be separable after being applied onto a surface. In embodiments, the layer containing the sulfonated polymer is optionally coated with an adhesive for the layer stick to surface, but is still releasable.

“Releasable” or “separable” bond in the context of layers or surfaces means that the layers or surfaces are generally attached or fastened to each other, yet can be separated with the application of a certain amount of force, and then subsequently refastened or reattached at a later time. In order to be “separable” or “releasable,” the surfaces must be capable of being fastened and separated, and the force applied to separate the layers or surfaces can be applied by hand.

“Solid” is in the context of a wound dressing refers to a gel, and not a liquid.

“Surface pH” refers to the pH on the contact surface of the bio-secure material, that results from surface bound moieties e.g., the coating layer. The surface pH can be measured with commercial surface pH measuring instruments, e.g., SenTix™ Sur-electrode from WTW Scientific-Technical Institute GmbH, Weilheim, Germany.

The disclosure relates to a wound dressing that kills microorganism within a predefined duration of contact with the antimicrobial layer. In embodiments, the wound dressing has a surface comprising, consisting essentially of, or consisting of a sulfonated polymer as a self-sterilizing (self-disinfecting) material. The surface comprising the sulfonated polymer can be in contact with the wound to be treated, as an outer layer opposite from the wound (for the protection of the wound from contamination.

Self-sterilizing Material—Sulfonated Polymer: Sulfonated polymer refers to polymers having a sulfonate group, e.g., —SO₃, either in the acid form (e.g., —SO₃H, sulfonic acid) or a salt form (e.g., —SO₃Na). The term “sulfonated polymer” also covers sulfonate containing polymers, e.g., polystyrene sulfonate.

The sulfonated polymer is selected from the group of perfluorosulfonic acid polymers (e.g., sulfonated tetrafluoroethylene), sulfonated polyolefins, sulfonated polyimides, sulfonated polyamides, sulfonated polyester, polystyrene sulfonates, sulfonated block copolymers, sulfonated polyolefins, sulfonated polysulfones such as polyether sulfone, sulfonated polyketones such as polyether ether ketone, sulfonated polyphenylene ethers, and mixtures thereof.

The sulfonated polymer is characterized as being sufficiently or selectively sulfonated to contain from 10-100 mol % sulfonic acid or sulfonate salt functional groups based on the number of monomer units or the block to be sulfonated (“degree of sulfonation”), to kill at least 95% of microbes within 120 minutes of coming into contact with the coating material. In embodiments, the sulfonated polymer has a degree of sulfonation of >25 mol %, or >50 mol %, or <95 mol %, or 25-70 mol %. Degree of sulfonation can be calculated by NMR or ion exchange capacity (IEC).

In embodiments, the sulfonated polymer is a sulfonated tetrafluoroethylene, having a polytetrafluoroethylene (PTFE) backbone; (2) side chains of vinyl ethers (e.g., —O—CF₂—CF—O—CF₂—CF₂—) which terminate in sulfonic acid groups in a cluster region.

In embodiments, the sulfonated polymer is a polystyrene sulfonate, examples include potassium polystyrene sulfonate, sodium polystyrene sulfonate, a co-polymer of sodium polystyrene sulfonate and potassium polystyrene sulfonate (e.g., a polystyrene sulfonate copolymer), having a molecular weight of 20,000 to 1,000,000 Daltons, or >25,000 Daltons, or >40,000 Dalton, or >50,000, or >75,000, or >100,000 Daltons, or >400,000 Daltons, or <200,000, or <800,000 Daltons, or up to 1,500,000 Daltons. The polystyrene sulfonate polymers can either be crosslinked or uncrosslinked. In embodiments, the polystyrene sulfonate polymers are uncrosslinked and water soluble.

In embodiments, the sulfonated polymer is a polysulfone, selected from the group of aromatic polysulfones, polyphenylenesulfones, aromatic polyether sulfones, dichlorodiphenoxy sulfones, sulfonated substituted polysulfone polymers, and mixtures thereof. In embodiments, the sulfonated polymer is a sulfonated polyethersulfone copolymer, which can be made with reactants including sulfonate salts such as hydroquinone 2-potassium sulfonate (HPS) with other monomers, e.g., bisphenol A and 4-fluorophenyl sulfone. The degree of sulfonation in the polymer can be controlled with the amount of HPS unit in the polymer backbone.

In embodiments, the sulfonated polymer is a sulfonated polyether ketone. In embodiments, the sulfonated polymer is a sulfonated polyether ketone ketone (SPEKK), obtained by sulfonating a polyether ketone ketone (PEKK). The polyether ketone ketone can be manufactured using diphenyl ether and a benzene dicarbonic acid derivative. The sulfonated

PEKK can be available as an alcohol and/or water-soluble product, e.g., for subsequent use to coat the face mask or in spray applications.

In embodiments, the sulfonated polymer is a sulfonated poly(arylene ether) copolymer containing pendant sulfonic acid groups. In embodiments, the sulfonated polymer is a sulfonated poly(2,6-dimethyl-1,4-phenylene oxide), commonly referred to as sulfonated polyphenylene oxide. In embodiments, the sulfonated polymer is a sulfonated poly(4-phenoxybenzoyl-1,4-phenylene) (S-PPBP). In embodiments, the sulfonated polymer is a sulfonated polyphenylene having 2 to 6 pendant sulfonic acid groups per polymer repeat, and characterized as having 0.5 meq (SO₃H)/g of polymer to 5.0 meq (SO₃H)/g polymer, or at least 6 meq/g (SO₃H)/g polymer.

In embodiments, the sulfonated polymer is a sulfonated polyamide, e.g. aliphatic polyamides such nylon-6 and nylon-6,6, partially aromatic polyamides and polyarylamides such as poly(phenyldiamidoterephthalate), provided with sulfonate groups chemically bonded as amine pendant groups to nitrogen atoms in the polymer backbone. The sulfonated polyamide can have a sulfonation level of 20 to up to 100% of the amide group, with the sulfonation throughout the bulk of the polyamide. In embodiments, the sulfonation is limited to a high density of sulfonate groups at the surface, e.g., >10%, >20%, >30%, or >40%, or up to 100% of the sulfonated amide group at the surface (within 50 nm of the surface).

In embodiments, the sulfonated polymer is a sulfonated polyolefin, containing at least 0.1 meq, or >2 meq, or >3 meq, or >5 meq, or 0.1 to 6 meq of sulfonic acid per gram of polyolefin. In embodiments, the sulfonated polymer is a sulfonated polyethylene. The sulfonated polyolefin can be formed by chlorosulfonation of a solid polyolefin obtained by polymerization of an olefin or a mixture of olefins selected from a group consisting of ethylene, propylene, butene-1,4-methylpentene-1, isobutylene, and styrene. The sulfonyl chloride groups can then be hydrolyzed, for example, in an aqueous base such as potassium hydroxide or in a water dimethylsulfoxide (DMF) mixture to form sulfonic acid groups. In embodiment, the sulfonated polyolefin is formed by submerging or passing polyolefin object in any form of powder, fiber, yarn, woven fabric, a film, a preform, etc., through a liquid containing sulfur trioxide (SO₃), a sulfur trioxide precursor (e.g., chlorosulfonic acid, HSO₃Cl), sulfur dioxide (SO₂), or a mixture thereof. In other embodiments, the polyolefin object is brought into contact with a sulfonating gas, e.g., SO₂ or SO₃, or gaseous reactive precursor, or a sulfonation additive that evolves a gas SOX at elevated temperature.

The polyolefin precursor to be sulfonated can be, for example, a poly-α-olefin, such as polyethylene, polypropylene, polybutylene, polyisobutylene, ethylene propylene rubber, or a chlorinated polyolefin (e.g., polyvinylchloride, or PVC), or a polydiene, such as polybutadiene (e.g., poly-1,3-butadiene or poly-1,2-butadiene), polyisoprene, dicyclopentadiene, ethylidene norbornene, or vinyl norbornene, or a homogeneous or heterogeneous composite thereof, or a copolymer thereof (e.g., EPDM rubber, i.e., ethylene propylene diene monomer). In embodiments, the polyolefin is selected from low density polyethylene (LDPE), linear low density polyethylene (LLDPE), very low density polyethylene (VLDPE), high density polyethylene (HDPE), medium density polyethylene (HDPE), high molecular weight polyethylene (HMWPE), and ultra-high molecular weight polyethylene (UHMWPE).

In embodiments, the sulfonated polymer is a sulfonated polyimide, e.g., aromatic polyimides in both thermoplastic and thermosetting forms, having excellent chemical stability and high modulus properties. Sulfonated polyimide can be prepared by condensation polymerization of dianhydrides with diamines, wherein one of the monomeric units contains sulfonic acid, sulfonic acid salt, or sulfonic ester group. The polymer can also be prepared by direct sulfonation of aromatic polyimide precursors, using sulfonation agents such as chlorosulfonic acid, sulfur trioxide and sulfur trioxide complexes. In embodiments, the concentration of sulfonic acid groups in the sulfonated polyimide as measured by ion exchange capacity, IEC, varying from 0.1 meq/g to above 3 meq/g, or at least 6 meq/g.

In embodiments, the sulfonated polymer is a sulfonated polyester, formed by directly sulfonating a polyester resin in any form, e.g., fiber, yarn, woven fabric, film, sheet, and the like, with a sulfuric anhydride-containing gas containing sulfuric anhydride, for a concentration of the sulfone group on the surface of the polyester ranging from 0.1 meq/g to above 3 meq/g, e.g., up to 5 meq/g, or at least 6 meq/g.

In embodiments, the sulfonated polymer is a selectively sulfonated negative-charged anionic block copolymer. The term “selectively sulfonated” definition to include sulfonic acid as well as neutralized sulfonate derivatives. The sulfonate group can be in the form of metal salt, ammonium salt or amine salt.

Depending on the applications and the desired properties, the sulfonated polymer can be modified (or funcationalized). In embodiments, the sulfonated polymer is neutralized with any of various metal counterions, including alkali, alkaline earth, and transition metals, with at least 10% of the sulfonic acid groups being neutralized. In embodiments, the sulfonated polymer is neutralized with inorganic or organic cationic salts, e.g, those based on ammonium, phosphonium, pyridinium, sulfonium and the like. Salts can be monomeric, oligomeric, or polymeric. In embodiments, the sulfonated polymer is neutralized with various primary, secondary, or tertiary amine-containing molecules, with >10% of the sulfonic acid or sulfonate functional groups being neutralized.

In embodiments, the sulfonic acid or sulfonate functional group is modified by reaction with an effective amount of polyoxyalkyleneamine having molecular weights from 140 to 10,000. Amine-containing neutralizing agents can be mono-functional or multi-functional; monomeric, oligomeric, or polymeric. In alternative embodiments, the sulfonated polymer is modified with alternative anionic functionalities, such as phosphonic acid or acrylic and alkyl acrylic acids.

In embodiments, amine containing polymers are used for the modification of the sulfonated polymers, forming members of a class of materials termed coaservates. In examples, the neutralizing agent is a polymeric amine, e.g., polymers containing benzylamine functionality. Examples include homopolymers and copolymers of 4-dimethylaminostyrene which has been described in U.S. Pat. No. 9,849,450, incorporated herein by reference. In embodiments, the neutralizing agents are selected from polymers containing vinylbenzylamine functionality, e.g., polymers synthesized from poly-p-methylstyrene containing block copolymers via a bromination-amination strategy, or by direct anionic polymerization of amine containing styrenic monomers. Examples of amine functionalities for functionalization include but are not limited to p-vinylbenzyldimethylamine (BDMA), p-vinylbenzylpyrrolidine (VBPyr), p-vinylbenzyl-bis(2-methoxyethyl)amine (VBDEM), p-vinylbenzylpiperazine (VBMPip), and p-vinylbenzyldiphenyl amine (VBDPA). In embodiments, corresponding phosphorus containing polymers can also be used for the functionalization of the sulfonated polymers.

In embodiments, the monomer or the block containing amine functionality or phosphine functionality can be neutralized with acids or proton donors, creating quaternary ammonium or phosphonium salts. In other embodiments, the sulfonated polymer containing tertiary amine is reacted with alkylhalides to form functional groups, e.g., quaternized salts. In some embodiments, the sulfonated polymer can contain both cationic and anionic functionality to form so-called zwitterionic polymers.

In some embodiments, the sulfonated polymer is a selectively sulfonated negative-charged anionic block copolymer, which “selectively sulfonated” definition to include sulfonic acid as well as neutralized sulfonate derivatives. The sulfonate group can be in the form of metal salt, ammonium salt or amine salt. In embodiments, the sulfonated block polymer has a general configuration A-B-A, (A-B)ₙ(A), (A-B-A)ₙX, (A-B)ₙX, A-D-B, A-B-D, A-D-B-D-A, A-B-D-B-A, (A-D-B)ₙA, (A-B-D)ₙA (A-D-B)ₙX, (A-B-D)ₙX or mixtures thereof; where n is an integer from 0 to 30, or 2 to 20 in embodiments; and X is a coupling agent residue. Each A and D block is a polymer block resistant to sulfonation. Each B block is susceptible to sulfonation. For configurations with multiple A, B or D blocks, the plurality of A blocks, B blocks, or D blocks can be the same or different.

In embodiments, the A blocks are one or more segments selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof. If the A segments are polymers of 1,3-cyclodiene or conjugated dienes, the segments will be hydrogenated subsequent to polymerization of the block copolymer and before sulfonation of the block copolymer. The A blocks may also contain up to 15 mol % of the vinyl aromatic monomers such as those present in the B blocks.

In embodiments, the A block is selected from para-substituted styrene monomers selected from para-methyl styrene, para-ethyl styrene, para-n-propyl styrene, para-iso-propyl styrene, para-n-butyl styrene, para-sec-butyl styrene, para-iso-butyl styrene, para-t-butylstyrene, isomers of para-decylstyrene, isomers of para-dodecylstyrene and mixtures of the above monomers. Examples of para-substituted styrene monomers include para-t-butylstyrene and para-methylstyrene, with para-t-butylstyrene being most preferred. Monomers may be mixtures of monomers, depending on the particular source. In embodiments, the overall purity of the para-substituted styrene monomers be at least 90%-wt., or >95%-wt., or >98%-wt. of the para-substituted styrene monomer.

In embodiments, the block B comprises segments of one or more polymerized vinyl aromatic monomers selected from unsubstituted styrene monomer, ortho-substituted styrene monomers, meta-substituted styrene monomers, alpha-methylstyrene monomer, 1,1-diphenylethylene monomer, 1,2-diphenylethylene monomer, and mixtures thereof. In addition to the monomers and polymers noted, in embodiments the B blocks also comprises a hydrogenated copolymer of such monomer (s) with a conjugated diene selected from 1,3-butadiene, isoprene and mixtures thereof, having a vinyl content of between 20 and 80 mol percent. These copolymers with hydrogenated dienes can be any of random copolymers, tapered copolymers, block copolymers or controlled distribution copolymers. The block B is selectively sulfonated, containing from about 10 to about 100 mol % sulfonic acid or sulfonate salt functional groups based on the number of monomer units. In embodiments, the degree of sulfonation in the B block ranges from 10 to 95 mol %, or 15-80 mol %, or 20-70 mol %, or 25-60 mol %, or >20 mol %, or >50 mol %.

The D block comprises a hydrogenated polymer or copolymer of a conjugated diene selected from isoprene, 1,3-butadiene and mixtures thereof In other examples, the D block is any of an acrylate, a silicone polymer, or a polymer of isobutylene with a number average molecular weight of >1000, or >2000, or >4000, or >6000.

The coupling agent X is selected from coupling agents known in the art, including polyalkenyl coupling agents, dihaloalkanes, silicon halides, siloxanes, multifunctional epoxides, silica compounds, esters of monohydric alcohols with carboxylic acids, (e.g. methylbenzoate and dimethyl adipate) and epoxidized oils.

The antimicrobial and mechanical properties of the sulfonated block copolymer can be varied and controlled by varying the amount of sulfonation, the degree of neutralization of the sulfonic acid groups to the sulfonated salts, as well as controlling the location of the sulfonated group(s) in the polymer. In embodiments and depending on the applications, e.g., one with the need for water dispersity/solubility, or at the other spectrum, one with the need for sufficient durability with constant wiping with water based cleaners, the sulfonated block copolymer can be selectively sulfonated for desired water dispersity properties or mechanical properties, e.g., having the sulfonic acid functional groups attached to the inner blocks or middle blocks, or in the outer blocks of a sulfonated block copolymer, as in U.S. Pat. No. 8,084,546, incorporated by reference. If the outer (hard) blocks are sulfonated, upon exposure to water, hydration of the hard domains may result in plasticization of those domains and softening, allowing dispersion or solubility.

The sulfonated copolymer in embodiments is as disclosed in Patent Publication Nos. U.S. Pat. Nos. 9,861,941, 8,263,713, 8,445,631, 8,012,539, 8,377,514, 8,377,515, 7,737,224, 8,383,735, 7,919,565, 8,003,733, 8,058,353, 7,981,970, 8,329,827, 8,084,546, 8,383,735, 10,202,494, and 10,228,168, the relevant portions are incorporated herein by reference.

In embodiments, the sulfonated block copolymer has a general configuration A-B-(B-A)₁₋₅, wherein each A is a non-elastomeric sulfonated monovinyl arene polymer block and each B is a substantially saturated elastomeric alpha-olefin polymer block, said block copolymer being sulfonated to an extent sufficient to provide at least 1% by weight of sulfur in the total polymer and up to one sulfonated constituent for each monovinyl arene unit. The sulfonated polymer can be used in the form of their acid, alkali metal salt, ammonium salt or amine salt.

In embodiments, the sulfonated block copolymer is a sulfonated polystyrene-polyisoprene-polystyrene, sulfonated in the center segment. In embodiments, the sulfonated block copolymer is a sulfonated t-butylstyrene/isoprene random copolymer with C═C sites in their backbone. In embodiments, the sulfonated polymer is a sulfonated SBR (styrene butadiene rubber) as disclosed in U.S. Pat. No. 6,110,616 incorporated by reference. In embodiments, the sulfonated polymer is a water dispersible BAB triblock, with B being a hydrophobic block such as alkyl or (if it is sulfonated, it becomes hydrophilic) poly(t-butyl styrene) and A being a hydrophilic block such as sulfonated poly(vinyl toluene) as disclosed in U.S. Pat. No. 4,505,827 incorporated by reference.

In embodiments, the sulfonated block copolymer is a functionalized, selectively hydrogenated block copolymer having at least one alkenyl arene polymer block A and at least one substantially completely, hydrogenated conjugated diene polymer block B, with substantially all of the sulfonic functional groups grafted to alkenyl arene polymer block A (as disclosed in U.S. Pat. No. 5,516,831, incorporated by reference). In embodiments, the sulfonated polymer is a water-soluble polymer, a sulfonated diblock polymer of t-butyl styrene/styrene, or a sulfonated triblock polymer of t-butyl styrene-styrene-t-butyl styrene as disclosed in U.S. Pat. No. 4,492,785 incorporated by reference. In embodiments, the sulfonated block copolymer is a partially hydrogenated block copolymer.

In embodiments, the sulfonated polymer is a midblock-sulfonated triblock copolymer, or a midblock-sulfonated pentablock copolymer or, e.g., a poly(p-tert-butylstyrene-b-styrenesulfonate-b-p-tert-butylstyrene), or a poly[tert-butylstyrene-b-(ethylene-alt-propylene)-b-(styrenesulfonate)-b-(ethylene-alt-propylene)-b-tert-butylstyrene.

In embodiments, the sulfonated polymer contains >15 mol %, or >25 mol %, or >30 mol %, or >40 mol %, or >60 mol % sulfonic acid or sulfonate salt functional groups based on the number of monomer units in the polymer that are available or susceptible for sulfonation, e.g., the styrene monomers.

In embodiments, the sulfonated polymer has an ion exchange capacity of >0.5 meq/g, or >0.75 meq/g, or >1.0 meq/g, or >1.5 meq/g, or >2.0 meq/g, or >2.5 meq/g, or <5.0 meq/g.

Optional Additives: In embodiments, the sulfonated polymer may also be blended with other polymers and used for preparing a suitable blend for deposition on the surface of the substrate by dipping, spraying, electrospraying, and electroaerosoling.

Further, the sulfonated polymer can be combined with any desired therapeutic agent to make the wound dressing more effective for the treatment of the wound.

In embodiments, the sulfonated polymer further contains or can be complexed with, or otherwise form mixtures, compounds, etc. with, antibiotics such as butylparaben and triclosan, e.g., antimicrobial surfactants, lipids, nanoparticles, peptides, antibiotics or antiviral drugs, quaternary ammonium and phosphonium containing polymers, chitosan and other naturally occurring antimicrobial polymers, ion-exchange resins, metallic-based micro and nano-structured materials such as silver, copper, zinc and titanium and their oxides, for enhanced antimicrobial effectiveness.

In embodiments, the sulfonated polymer further comprises additives that wound help signal or give an indicator of its antimicrobial effects with a color change pH indicator. Examples include Thymol Blue, Methyl Orange, Bromocresol Green, Methyl Red, Bromothymol

Blue, Phenol Red, and Phenol-phthalein. A color change means a change in hue, from a light to a darker color or vice versa. A color indicator may indicate if a recharge, regeneration, or reactivation of the antimicrobial activity of the protective layer is recommended. The color indicator is incorporated in a sufficient amount so that a noticeable change in color hue is observed immediately when there is a change in the effectiveness of the sulfonated polymeric material, e.g., when its surface pH is dropped below 2.0, or when its ion exchange capacity (IEC) falls below the desired level for the antimicrobial application. In embodiments, the amount of color indicator ranges from 0.1 to 20 wt. % of the amount of sulfonated polymer applied as a protective layer on at least one surface of the substrate of the wound dressing. The sulfonated polymer comprising the color indicator to indicate the need of change of the wound dressing with another unused/new wound dressing.

In addition to the above optional components, other additives such as plasticizers, tackifiers, surfactants, film forming additives, dyes, pigments, cross-linkers, UV absorbers, catalysts, highly conjugated particles, or tubes (e.g. carbon black, graphene, carbon nanotubes), etc. may be incorporated in any combination to the extent that they do not reduce the efficacy of the material.

Properties of Sulfonated Polymer: In embodiments, the sulfonated polymer is characterized as being sufficiently sulfonated to have an IEC of >0.5 meq/g, or 1.5-3.5 meq/g, or >1.25 meq/g, or >2.2 meq/g, or >2.5 meq/g, or >4.0 meq/g, or <4.0 meq/g.

In embodiments, the sulfonated polymer is characterized as having a surface pH of <3.0. It is believed that a sufficiently low surface level, as a result of the presence of sulfonic acid functional groups in the protective layer, wound have catastrophic effects on microorganisms that come in contact with the surface of the substrate of the wound dressing.

In embodiments, the sulfonated polymer works effectively in destroying/inactivating at least 99%, or at least 99.5%, or at least 99.9% of microorganisms in <30 minutes of exposure, or <5 minutes of exposure or contact with microorganisms, including but not limited to MRSA, vancomycin-resistant Enterococcus faecium, X-MulV, PI-3, SARS-CoV-2, carbapenem-resistant Acinetobacter baumannii, and influenza A virus. In embodiments with polymer containing quaternary ammonium group, the material is effective in killing target microorganisms including Staphylococcus aureus, Escherichia coli, Staphylococcus albus, Escherichia coli, Rhizoctonia solani, and Fusarium oxysporum. The sulfonated polymer remains effective in killing microbes even after 4 hours, or after 12 hours, or at least 24 hours, or for at least 48 hours.

In embodiments, the sulfonated polymer is a sulfonated block copolymer, e.g., a midblock-sulfonated pentablock copolymer, containing >40 mol % sulfonic acid or sulfonate salt functional groups based on the number of monomer units. In tests simulating cleaning of the surface of a sulfonated polymeric film, after 2400 cleaning or abrasion cycle, representing 200 days (at least 6 months) in use with 6 cleaning sessions per day (with 4 rubbing motions per session with alcohol and/or quaternary ammonium compounds cleaners).

Methods for Applying/Incorporating Sulfonated Polymers onto/into Wound Dressing: The wound dressing comprises a substrate for the protection of the wound, with the sulfonated polymer as a protective layer or coating for at least one side of the substrate, or for the sulfonated polymer to be impregnated onto the wound dressing substrate (e.g., a gauze or a bandage). In embodiments, the wound dressing is in the form of a gel, with the sulfonated polymer being incorporated into the gel, or as a protective layer on the gel.

The sulfonated polymer can be applied onto the surface of the substrate before or after it is made, or it is incorporated into the wound dressing as one of the components. In embodiments, the sulfonated polymer is as a protective coating on at least one surface of the substrate of the wound dressing, or as a self-adhesive protective film, or coated onto a pressure sensitive adhesive layer (e.g., a peel and stick film) for protecting the surface of the substrate and the sulfonated polymer layer. In embodiments, the sulfonated polymer is applied as a protective layer having a thickness of <1000 μm, or >1 μm, or >5 μm, or >10 μm, or <500 μm, or <200 μm, or <100 μm, providing a self-sterilizing surface.

In embodiments, the sulfonated polymer is first electrospun (e-spun), generating nanoscale to microscale fibers with disinfecting properties. In electrospinning, a solution comprising the sulfonated polymer is fed to a multi-nozzle device or a nozzle free device, and a high voltage is applied. The solution is transformed under the influence of the high voltage into charged jet streams and deposited onto a substrate, or take by a collector. The polymer in the jet stream solidifies thereby forming nanofibers or microfibers. Electrospinning can be used to generate sulfonated microfibers or nanofibers of less than 400 nm, or less than 200 nm, or 50-300 nm, or less than 250 microns, or 50-150 microns, or 40-90 microns, depending on the specific electrospinning conditions employed.

In embodiments, the sulfonated polymer in solution is e-spun via a multi-nozzle device or a nozzle-free electrospinning with the use of nozzle free device onto layers of wound dressing (e.g., a gauze sheet, a plurality of fibrous layers) as laid out, e.g., in a clean room conveyor belt. In embodiments, the amount of sulfonated polymers (or density) of e-spun sulfonated block copolymer on the wound dressing ranges from 1-30 grams per square meter per mil thickness, or 2-10 g/m², or at least 3 g/m², or less than 5 g/m², for an electrospun sulfonated polymer mat having a thickness of <500 nm, <200 nm, or <100 nm, or at least 50 nm.

By controlling factors including but not limited to the concentration of the sulfonate polymer in solution, the diameter of the nozzle, the speed of the melt spinning process, the speed the conveyor belt, the spinning distance, and the applied voltage, the thickness and amount of electrospun sulfonated nanofibers or microfibers can be controlled to provide sufficient coverage of the surface of the wound dressing. The wound dressing, the gauze sheet, or roll coated with the sulfonated polymer can be subsequently cut into sizes for packaging/sold to be used as air filters.

In embodiments, the electrospun sulfonated microfibers or nanofibers form a stand-alone nanofiber or microfiber layer or mat, having a thickness of <500 nm, <200 nm, or <100 nm, or at least 50 nm, for use with the wound dressing or gauze, and supported by the gauze made of a different material, e.g., medical fabric made from the fibers of cotton, polyester, rayon, etc. Multiple gauze layers can be used, sandwiched or alternating with layers comprising the sulfonated polymer.

In embodiments, the electrospun sulfonated microfibers or nanofibers are interweaved with other fibers, e.g., microfibers, submicron fibers and nanofibers from other materials, e.g., polyester, rayon, cotton, etc., forming a wound dressing. Depending on the sulfonated polymer employed, in embodiments in addition to self-sterilizing properties, the sulfonated polymer also facilities or controls the vapor transmission rate and provides an environment that would accelerate wound healing.

Examples of other different polymers for use in the filter medium include but are not limited to cellulose acetate (CA), polyolefin, polyamide 6 (PA 6), polystyrene (PS), polyacrylonitrile (PAN), polyvinylpyrrolidone (PVP), polyvinyl alcohol (PVA), polyethylene oxide (PEO), poly(lactic acid) (PLA), poly(lactic-co-glycolic acid) (PLGA), polybutylene terephthalate (PBT) and polyurethane (PU), or natural polymers such as gelatin, chitosan and polyhydroxybutyrate-co-hydroxyvalerate (PHBV).

In embodiments, electrospun sulfonated microfibers or nanofibers are interweaved with other fibers, e.g., microfibers, submicron fibers and nanofibers from other different polymers, forming a self-sterilizing wound dressing layer. Depending on the sulfonated polymer employed, in embodiments in addition to self-sterilizing properties, the polymer also facilities or controls the vapor transmission rate and provides an environment that would accelerate wound healing. In embodiments, the electrospun fibers containing sulfonated polymer are made into a stand-alone fibrous layer, providing an environment on the surface of a wound to kill microbes, and a barrier against microbe penetration, preventing the wound from an infection. The stand-alone sulfonated polymer fibrous layer is used in conjunction with other polymeric layers (as support or substrate) forming a wound dressing.

Examples of other different polymers include but are not limited to cellulose acetate (CA), polyolefin, polyamide 6 (PA 6), polystyrene (PS), polyacrylonitrile (PAN), polyvinylpyrrolidone (PVP), polyvinyl alcohol (PVA), polyethylene oxide (PEO), poly(lactic acid) (PLA), poly(lactic-co-glycolic acid) (PLGA), polybutylene terephthalate (PBT) and polyurethane (PU), or natural polymers such as gelatin, chitosan and polyhydroxybutyrate-co-hydroxyvalerate (PHBV).

In embodiments, the wound dressing is provided with an adhesive layer. Wherein the adhesive layer provides a gentle adhesion to the wound and the surround skin. Preferably, the adhesive layer is a pressure sensitive adhesive layer having silicone, acrylate, and hot melt based adhesive.

In embodiments, a suitable primer layer may be provided between the surface of the substrate and the adhesive layer. The primer layer may comprise suitable primer such as silicone based to enhance the adhesion between the surface and the adhesive layer.

In embodiments, the substrate of the wound dressing may be chosen from but not limited to natural polymers such as carboxymethyl cellulose, cellulose acetate, pectin, xanthan gum, polysaccarides, alginates, chitosan, marine algae extract, polyaspartic acid, polyglutamic acid. The substrate may also be selected from synthetic polymers but not limited to polyolefins, polyurethanes, polyethylene vinyl acetate, polystyrene, polybutadiene, polyacrylate, polyurethane, polyisoprene, polycaprolactone, polylactic acid, polyamides, polyamine, polyaniline, polyester, silicone, copolymers thereof, blends of natural polymers, blends of synthetic polymers, and blends of natural and synthetic polymers can be applied as the substrate in preparation of wound dressing.

In embodiments, the substrate comprising natural or synthetic polymers can be in the form of a gauze, film, patches, a sheet, a multi-layer assembly, a fabric, a textile, a woven or non-woven fibers, a meltblown web, a spunbond web, and laminates and of any size, and shape to prepare the wound dressing for treating the wound. The thickness of the substrate is such that it can be flexible or stiff based on the requirement of treatment of the wound.

In embodiments, the sulfonated polymer material is dispersed in a solvent in an amount up to 10 wt. %, or up to 20 wt. %, or up to 50 wt. %, for coating the substrate as a protective layer. Depending on the sulfonated polymer used, exemplary solvents include but are not limited to water, isopropyl alcohol, acetone, N,N-dimethylacetamide, 1-methyl-2-pyrrolidinone, 1,3-dioxolane, 2-methoxy ethanol, dimethylformamide, or benzyl alcohol. In embodiments, the sulfonated polymer is applied by preparing a solution of the polymer in a suitable solvent, then casting on the surface of substrate to be subsequently formed into the wound dressing, with the thickness of the protective polymeric layer being adjusted with a casting knife, followed by drying.

In embodiments, with the wound dressing being in the form of a gel (e.g., hydrogel sheet dressing), the sulfonated polymer is applied onto the gel by any of spray coating, dipping, and the like. The gel wound dressing can be a composite material having a fibrous substrate, e.g., cotton gauze impregnated with a gel-forming polymer. The gel wound dressing in embodiments consists essentially of gel materials, e.g., a gel polymer in sheet form reinforced with gel-forming fibers.

In embodiments, the protective sulfonated polymeric layer is formed on the surface of the substrate for use in wound dressing by methods including but not limited to spray coating, or dip coating the substrate into a solution or dispersion containing the sulfonated polymer. Multiple coatings can be applied sequentially.

In embodiments, the sulfonated polymeric layer is applied on the surface of the substrate as a peel-and-stick film. The peel-and-stick film is first peeled off to remove an optional support/release liner if present, and then applied directly onto the surface as a protective layer.

In embodiments, the wound dressing is removable without leaving any residue of the adhesive layer or the sulfonated polymeric layer on application to the wound area.

In embodiments, the sulfonated polymer can be grafted, or covalently bonded onto the surface of the substrate. It will be further understood that the sulfonated polymer layer may be secured to the surface of the substrate by means of mechanical fixation with elements such as stitches, pins, or staples.

In embodiments, the sulfonated polymer is incorporated into a biodegradable polymer for forming the wound dressing, e.g., a suture, a hydrogel dressing or an alginate dressing, in an amount of 0.10 to 20 wt. %, or >0 25 wt %, or >0.5%, or >1.0%, or <15 wt. %, or <10 wt. %, or <5 wt. %, or <2 wt %. based on the total weight of the polymer blend The biodegradable polymers upon undergoing degradation can be consumed by the body without any undesirable side effects. The biodegradable polymer in embodiments is selected from the group of poly lactic-glycolic acid (PLGA), poly-caprolactonc (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalatc (PEO-PBTP), poly-D,L-lactic acid-p-dioxanone-poiyethylene glycol block copolymer (PLA-DXPEG), and combinations thereof In embodiments, the natural biodegradable polymer is selected from polysaccharides such as alginate, dextran, cellulose, collagen, and chemical derivatives thereof. After being incorporated into the biodegradable polymer, the composition can be blown, extruded into sheets or shaped by injection molding, or electrospun, for example, forming fibers for subsequently mads into sutures, or for forming non-woven nanofibrous mats.

Example 1: Tests were conducted to evaluate antimicrobial efficacy & the long-lasting antiviral properties of sulfonated polymers, film samples of sulfonated penta block copolymer (SPBC) of the structure poly[tert-butylstyrene-b-(ethylene-alt-propylene)-b-(styrene-co-styrene-sulfonate)-b-(ethylene-alt-propylene)-tert-butylstyrene] with 52% sulfonation were cast out of 1:1 mixture of toluene and 1 -propanol. The sulfonated polymer film samples were subjected to abrasion testing of 2200 cycles in the presence of 3 common disinfectants: 1) 70% ethanol, benzalkonium chloride, and quaternary ammonia], and exposure to SARS-CoV-2 virus suspension of concentration 107 pfu/ml.

After 2 hours of contact, viable virus was recovered from each sample by washing twice with 500 μl of DMEM tissue culture media containing 10% serum, and measured by serial dilution plaque assay. Gibco Dulbecco's Modified Eagle Medium (DMEM) is a basal medium for supporting the growth of many different mammalian cells. The results demonstrate that, after abrasion testing representing approximately one year of cleaning (6 disinfectant wipes/day), surface pro Gibco Dulbecco's Modified Eagle Medium (DMEM) is a widely used basal medium for supporting the growth of many different mammalian cells.

Example 2: The example was conducted to evaluate the effectiveness in inhibiting Aspergillus niger black mold according to the A ATCC Test Method 30-2004 Test III. Six different sulfonated block copolymer membrane samples comprising a poly[tert-butylstyrene-b-(ethylene-alt-propylene)-b-(styrenesulfonate)-b-(ethylene-alt-propylene)-b-tert-butylstyrene], at different levels of sulfonation from 26 to 52% were used for the tests. Aspergillus niger, ATCC#6275, was harvested into sterile distilled water containing glass beads. The flask was shaken to bring the spores into suspension The suspension was used as the test inoculum. One (1.0) mL of the inoculum was even distributed over the surface of Mineral Salts Agar plates The membrane samples were placed onto the inoculated agar surface. After placement, 0.2 mL of the inoculum was distributed over the surface of each disc. A viability plate of the spore suspension was prepared on Mineral Salts Agar with 3% glucose. A positive growth control was prepared using an untreated cotton duck fabric on Mineral Salts Agar and set up in the same manner as the test items. All samples were incubated at 28° C.±1° C. for 14 days.

The viability plate had acceptable fungal growth as expected confirming the viability of the inoculum. The sample with 26% sulfonation showed microscopic growth on 10% of the sample surface. The other 5 test samples showed no growth, or microscopic growth on 1% of the surface. The control sample showed macroscopic growth on 100% of the surface.

Example 3: Woven fabric of nylon 6, 6 fibers is immersed for 5 minutes in a solution of 0.5 g potassium t-butoxide and 0.5 g methanol in 10 ml of DMSO to provide deprotonated amines on the amide nitrogen in the polymer backbone. The deprotonated polymer is immersed in a solution of 0.33 g of sodium 4-bromobenzylsulfonic acid in 3.3. g DMSO (52° C.) to provide a fabric of polyamide fibers having benzylsulfonate groups attached to the surface thereof. The fabric of sulfonated polyamide fiber is washed with deionized (DI) water and dried to provide a fabric that can be made into gauze for use in wound dressings.

Example 4: A sulfonated polyester fabric is prepared, for use in making face masks, protective clothing, and the like. First a polyester taffeta made of polyester fiber is put into an acid-resistant sealable container. Sulfuric anhydride previously diluted 10 times with nitrogen gas is brought into contact with the polyester cloth for a sulfonated polyester material. The cloth is then washed with water and dried to produce a sulfonated polyester fabric, for use in making wound dressings, or a wound contact layer.

As used herein, the term “comprising” means including elements or steps that are identified following that term, but any such elements or steps are not exhaustive, and an embodiment can include other elements or steps. Although the terms “comprising” and “including” have been used herein to describe various aspects, the terms “consisting essentially of” and “consisting of” can be used in place of “comprising” and “including” to provide for more specific aspects of the disclosure and are also disclosed.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained. It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items.

Unless otherwise specified, all technical and scientific terms used herein have the same meanings as commonly understood by one of skill in the art to which the disclosed disclosure belongs. the recitation of a genus of elements, materials or other components, from which an individual component or mixture of components can be selected, is intended to include all possible sub-generic combinations of the listed components and mixtures thereof.

The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. To an extent not inconsistent herewith, all citations referred to herein are hereby incorporated by reference.

1. A self-sterilizing wound dressing comprising:
a substrate having a first surface facing at least a portion of a wound or a surgical site and a second surface facing opposite to the first surface,
wherein at least one of the first surface and the second surface of the substrate comprises a sulfonated polymeric layer having a thickness of at least >1μm for killing at least 90% of microbes coming in contact with the sulfonated polymeric layer within <120 minutes;
wherein the sulfonated polymeric layer consists essentially of a sulfonated polymer, the sulfonated polymer is selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof, wherein the sulfonated polymer has a degree of sulfonation of at least 10%.

2. The self-sterilizing wound dressing of claim 1, wherein the sulfonated polymer has an ionic exchange capacity (IEC) of >0.5 meq/g.

3. The self-sterilizing wound dressing of claim 1, wherein the sulfonated polymeric layer has a thickness of at least >5 μm to kill >95% of microbes within 30 minutes of contact.

4. The self-sterilizing wound dressing of claim 1, wherein the sulfonated polymer is selectively sulfonated to contain from 10-100 mol % sulfonic acid or sulfonate salt functional groups based on the number of monomer units or blocks in the sulfonated polymer susceptible to sulfonation, for the coating material to kill at least 95% of microbes within 30 minutes of contact.

5. The self-sterilizing wound dressing of claim 1, wherein the sulfonated polymer is a selectively sulfonated negative-charged anionic block copolymer having a general configuration of: A-B-A, (A-B)ₙ(A), (A-B-A)ₙX, (A-B)ₙX, A-D-B, A-B-D, A-D-B-D-A, A-B-D-B-A, (A-D-B)ₙA, (A-B-D)ₙA (A-D-B)ₙX, (A-B-D)ₙX or mixtures thereof, wherein
n is an integer from 0 to 30,
X is a coupling agent residue,
each A and D block is a polymer block resistant to sulfonation,
each B block is susceptible to sulfonation,
the A block is selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof;
the B block is a vinyl aromatic monomer, and
the D block is a hydrogenated polymer or copolymer of a conjugated diene selected from isoprene, 1,3-butadiene and mixtures thereof; and
wherein the block B is selectively sulfonated to contain from 10 - 100 mol % sulfonic acid or sulfonate salt functional groups based on the number of monomer units, for the coating material to kill at least 99% of microbes within 30 minutes of contact.

6. The self-sterilizing wound dressing of claim 1, wherein the sulfonated polymeric layer has a surface pH of <3.0.

7. The coating material self-sterilizing wound dressing of claim 1, wherein the sulfonated polymer is neutralized with a salt selected from ammonium, phosphonium, pyridinium, and sulfonium salts.

8. The self-sterilizing wound dressing of claim 1, wherein the sulfonated polymeric wherein the sulfonated polymer is a selectively sulfonated negative-charged anionic block copolymer, having at least one alkenyl arene polymer block A and at least one substantially completely, hydrogenated conjugated diene polymer block B, with substantially all of the sulfonic functional groups grafted to alkenyl arene polymer block A for the block A to be a hydrophilic end-block.

9. The self-sterilizing wound dressing of claim 1, wherein both the first surface and the second surface of the substrate comprise the sulfonated polymeric layer for killing at least 99% microorganisms within 30 minutes of contact with the sulfonated polymeric layer.

10. The self-sterilizing wound dressing of claim 1, further comprising an adhesive layer coupled between the first surface of the substrate and the sulfonated polymeric layer, wherein the adhesive layer is partially or completely coated by the sulfonated polymeric layer.

11. The self-sterilizing wound dressing of claim 1, wherein the substrate being selected from the group of natural polymer, synthetic polymer or mixtures thereof.

12. The self-sterilizing wound dressing of claim 11, wherein natural polymer being selected from the group of carboxymethyl cellulose, cellulose acetate, pectin, xanthan gum, polysaccarides, alginates, chitosan, marine algae extract, polyaspartic acid, polyglutamic acid and wherein synthetic polymer being selected from polyolefins, polyurethanes, polyethylene vinyl acetate, polystyrene, polybutadiene, polyacrylate, polyurethane, polyisoprene, polycaprolactone, polylactic acid, polyamides, polyamine, polyaniline, polyester, silicone, copolymers thereof and blends thereof.

13. The self-sterilizing wound dressing of claim 1, wherein the substrate is any of fabric, textile, non-wovens, protective garment, a meltblown web, a spunbond web, a suture, and laminates.

14. The self-sterilizing wound dressing of claim 1, wherein the substrate is a suture, and wherein the suture is formed by electrospinning a blend of the sulfonated polymer and at least a biodegradable polymer selected from the group of polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), polysaccharides and derivatives thereof, and combinations thereof.

15. A method for treating a wound, the method comprises applying onto the wound or a cut the wound dressing of claim 1.

16. A self-sterilizing wound dressing comprising:
a substrate having a first surface facing at least a portion of a wound or a surgical site and a second surface facing opposite to the first surface,
wherein at least one of the first surface and the second surface of the substrate comprises a sulfonated polymeric layer having a thickness of at least >1 μm for killing at least 90% of microbes coming in contact with the sulfonated polymeric layer within <120 minutes;
wherein the sulfonated polymeric layer consists essentially of a sulfonated polymer, the sulfonated polymer is selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof, wherein the sulfonated polymer has a degree of sulfonation of at least 10%;
an adhesive layer coupled between the first surface of the substrate and the sulfonated polymeric layer, wherein the adhesive layer is partially or completely coated by the sulfonated polymeric layer.

17. The self-sterilizing wound dressing of claim 16, wherein the sulfonated polymer has an ionic exchange capacity (IEC) of >0.5 meq/g.

18. The self-sterilizing wound dressing of claim 16, wherein the sulfonated polymeric layer has a thickness of at least >5 μm to kill >95% of microbes within 30 minutes of contact.

19. The self-sterilizing wound dressing of claim 16, wherein the sulfonated polymer is selectively sulfonated to contain from 10-100 mol % sulfonic acid or sulfonate salt functional groups based on the number of monomer units or blocks in the sulfonated polymer susceptible to sulfonation, for the coating material to kill at least 95% of microbes within 30 minutes of contact.

20. The self-sterilizing wound dressing of claim 16, wherein the sulfonated polymer is a selectively sulfonated negative-charged anionic block copolymer having a general configuration of: A-B-A, (A-B)ₙ(A), (A-B-A)ₙX, (A-B)ₙX, A-D-B, A-B-D, A-D-B-D-A, A-B-D-B-A, (A-D-B)ₙA, (A-B-D)ₙA (A-D-B)ₙX, (A-B-D)ₙX or mixtures thereof, wherein
n is an integer from 0 to 30,
X is a coupling agent residue,
each A and D block is a polymer block resistant to sulfonation,
each B block is susceptible to sulfonation,
the A block is selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof;
the B block is a vinyl aromatic monomer, and
the D block is a hydrogenated polymer or copolymer of a conjugated diene selected from isoprene, 1,3-butadiene and mixtures thereof; and
wherein the block B is selectively sulfonated to contain from 10-100 mol % sulfonic acid or sulfonate salt functional groups based on the number of monomer units, for the coating material to kill at least 99% of microbes within 30 minutes of contact.
